(19) United States
(12) Patent Application Publication
(10) Pub. No.: US 2003/0087289 A1
(43) Pub. Date: May 8, 2003

(54) Image analysis of high-density synthetic DNA microarrays

(76) Inventors: Harry Zuzan, Pleasant Hill, CA (US); Valen E. Johnson, Ann Arbor, MI (US)

Correspondence Address: MYERS BIGEL SIBLEY & SAJOVEC, PO BOX 37428, RALEIGH, NC, 27627, US

(21) Appl. No.: 10/261,570

(22) Filed: Sep. 30, 2002

Related U.S. Application Data
(60) Provisional application No. 60/329,023, filed on Oct. 12, 2001.

(51) Int. Cl. C12Q 1/68; G06F 19/00; G01N 15/08; G01N 33/48; G01N 33/50; G06K 9/00

(52) U.S. Cl. 435/6; 702/20; 382/128

(57) ABSTRACT

Methods, systems, and computer program products for analyzing images of high density microarray chips analyze the image by estimating background using a blurring kernel and/or a spatial multivariate statistical model of the background. The methods, systems, and computer program products can employ a multivariate statistical model and/or a blurring kernel to obtain more representative hybridization intensity results, particularly for pixels in boundary regions of the probe cells. The methods allow for alternative microarray configurations of nucleic acid probes and do not require the use of mismatch probes and can be independent of the type of nucleotide sequence used. Associated microarrays and systems are also described.

### RELATED APPLICATIONS

This application claims priority from U.S. Provisional Patent Application Serial No. 60/329,023, filed Oct. 12, 2001, the contents of which are hereby incorporated by reference as if recited in full herein.

### FIELD OF THE INVENTION

The present invention relates to methods for analyzing images of biomaterial microarrays such as a High Density Synthetic-oligonucleotide DNA Microarray (“HDSM”).

### BACKGROUND OF THE INVENTION

Rapid extraction of gene expression data from DNA microarrays or microchips can provide researchers important information regarding biological processes. One type of array or chip used to obtain gene expression data is a HDSM. One commercially available chip is called a GeneChip® manufactured by Affymetrix, Inc. of Santa Clara, Calif.

Technology used to produce HDSM's have now miniaturized the area of the surface area used to hybridize an RNA sample to DNA probes. For example, one HSDM may contain about 300,000-400,000 (or more) different DNA probe sequences for a single hybridization, all within relatively small size, such as a 1.28 cm×1.28 cm region (hence, the term “microarray”). Redundant copies of each DNA sequence are located on the chip or array within a region termed a probe cell. Thus, typical HDSM's include about 300,000-400,000 probe cells. See Lockhart et al., Expression monitoring by hybridization to high-density oligonucleotide arrays, 14 Nature Biotechnology, pp. 1675-1680 (1996); and Lipshutzet al., High density synthetic oligonucleotide arrays, 21 Nature Genetics, pp. 20-24 (1999). In this miniaturized chip, it is possible to detect gene expression in a sample of RNA using approximately 400,000 different DNA probe sequences in a single simultaneous hybridization. This single simultaneous hybridization can be described as a parallel acquisition of data. This parallel methodology can reduce temporal sources of experimental error with respect to the hybridization process and/or data acquisition. A single RNA sample can be sufficient for an entire parallel hybridization and this can reduce the sources of error due to treatment levels from the evaluation process.

Generally described, hybridizations on the HSDM take place on a glass support, which is an impermeable rigid substrate which may reduce the variability in the observed outcome which may be introduced by porous substrates. Examples of variable conditions which may influence the observed outcome of the hybridization on the HSDM's include the quantity of RNA hybridized and measurement error in quantifying the RNA hybridized during the data acquisition process. See, e.g., Southern et al., Molecular interactions on microarrays, 21 Nature Genetics, pp. 5-9 (1999).

In operation, a sample of a fluorescent-labeled RNA or DNA hybridized to DNA probes on the HSDM is represented by an optically detectable fluorescence. The hybridization data is extracted by an image system which may use laser confocal fluorescence scanning which can be recorded as a large array of 16 bit integers to record the intensity of the image.

Operatively, an image-processing algorithm is used to define or estimate the location of each probe cell within the raw image. That is, the raw image data of intensity of the expressed chip is to be contrasted with the expressed (HSDM) chip itself (the intensity image does not itself define the location of the probe cells which physically reside on the chip). The HSDM is a segmented object. The glass substrate on which the probe cells are laid out can be partitioned into a contiguous array of probe cells surrounded by a border region. Thus, there is one segment for each probe cell and one segment for the border area. Any point on the glass support may be considered to be either interior to a probe cell or interior to the border region surrounding the array of probe cells.

The extracted image of the array can be presented as a grayscale image where each pixel maps to a small region of the HSDM. Generally stated, uniformly spaced probe cells can be arranged in a rectangular grid. In some designs of HSDM's, each probe cell occupies an area which is approximately 8×8 pixels in the image. These 8×8 pixel areas correspond to a physical area on the HSDM itself of about 21.5 μm×25 μm. Designs where probe cells on the HSDM occupy smaller areas result in probe cells occupying smaller regions of pixels in the HSDM image.

However, when obtaining the image, which pixels belong to which probe cells is not known prior to scanning. Allocation of pixels to probe cells is performed as a post-processing step on the extracted image data. Generally stated, each pixel in the HSDM image represents a small area on the actual HSDM surface. This area could be interior to a probe cell. It could straddle as many as four probe cells. It could also be partly or entirely in the border region surrounding the array of probe cells. Evident in many HSDM images is the effect of a blurring process. Hence, while the image is extracted, each pixel can accumulate signal not only from the area of the HSDM it represents on the surface of the chip but also from a small surrounding region. By the same blurring process, each pixel may lose signal to pixels nearby. Due to the discrete approximation of the HSDM surface provided by pixels and the effect of the blurring process, an HSDM image may not be viewed as a segmented collection (segmented by probe cells) even though the physical HSDM surface is.

Intensities of pixels representing areas on or near the perimeter of probe cells may be affected by the lack of image segmentation, in the sense that these intensities may not represent signal accumulated from a single probe cell. Further, probe cells representing genes that are not expressed should have low pixel intensities (such as “zero”), but there is evidence to suggest that a non-zero and non-constant background illumination and/or noise generated during the interrogation or image acquisition may undesirably contribute to pixel intensities in the image. Unfortunately, spatially variable intensity associated with artifacts, background and/or noise within probe cells and/or over many probe cells may impact the reproducibility or analysis of results.

In view of the above, there remains a need for improved image analysis methods that can evaluate images of expressed DNA microarrays.

### SUMMARY OF THE INVENTION

Embodiments of the present invention provide methods, systems, and computer program products for improved image analysis of DNA microarrays that can account for background illumination or other abnormalities that may be present in an HDSM image. Embodiments of the present invention also provide hybridization ranking methods and computer program products and microarray probe configurations.

Embodiments of the present invention provide alternative analysis methodologies to those intended by the design of the microarray by not requiring the use of mismatch probe sequences as controls. Embodiments of the present invention facilitate ranking methods applied to perfect match probes only, and thus, permit the non-parametric ranking Rank Sum Test as applied to perfect match probes only. Other embodiments of the present invention permit analysis of mismatch probes. Thus, the present invention contemplates analysis of any subset of the probes regardless of whether they are perfect match probes or mismatch probes, and irrespective of what gene they belong to. In operation, the present invention provides methods that estimate the background noise to define “zero.” Then, any subset of probes can be analyzed to generate an appropriate classification procedure. For example, for a random sample of probes, a classification can be based on the patterns in this random sample. These patterns are sometimes described by those of skill in the art as “fingerprints.” A search of a subset of probes can yield fingerprints which classify well.

Certain embodiments of the invention are directed to a method, system or computer program products for evaluating an image of a hybridized microarray. An image of a microarray having a plurality of individual probe cells is obtained. First estimated intensity values of pixels in the image are determined. The background intensity values for the pixels are estimated based on a predetermined multivariate statistical model. The second estimated intensity values of pixels in the image are determined by correcting the first estimated values to account for the estimated background intensity values.

In certain embodiments, the statistical model incorporates a Markov random field to model the spatial correlation of the background noise. The model may also be defined so as to incorporate a blurring kernel so that the estimation considers the intensity values of pixels in neighboring probe cells. In certain other embodiments, the statistical model can also or alternatively with the Markov random field include a blurring kernel which can be used to deconvolute the blurred probe cells in the image to thereby represent the intensity (a more accurate or truer intensity) of the fluorescence over substantially the entire probe cell.

In certain embodiments of the present invention, the image of an expressed microarray can be evaluated by obtaining an image of an expressed microarray having a plurality of individual probe cells and estimating the regions of the locations of each probe cell undergoing analysis in the image. Each probe cell location and proximate surrounding region includes a plurality of associated pixels affected by the fluorescence or lack of fluorescence of the respective probe cell. First estimated pixel intensity values for pixels in each probe cell region are determined. The intensity of background illumination is estimated for each pixel in the image to estimate spatial distribution of background intensity over the image. For each pixel in the image, the first estimated pixel intensity value is reduced to a second estimated pixel intensity based on data provided by the estimated background (thus reducing the first estimated pixels intensities in each probe cell region).

In certain embodiments, the analysis considers whether the background intensity undergoes abrupt changes or assumes an undesirable realization provided by the model to assess whether there is an abnormality and/or to identify the presence of an artifact or unreliable probe data.

In certain other embodiments, an image of an HSDM can be evaluated by obtaining an image of an HSDM having a plurality of individual probe cells and estimating the location of each probe cell undergoing analysis in the image. First estimated pixel intensity values can be obtained for a plurality of pixels in the region of each probe cell. Background intensity is estimated for each probe cell to obtain a spatial distribution of background intensity. A second estimated pixel intensity value for each probe cell can be determined by reducing the first estimated pixel intensity value by its corresponding estimated background intensity.

Still other embodiments of the present invention evaluate data obtained from an image of an hybridized microarray by analyzing image data corresponding to a probe cell location in the image to deconvolute the spread of fluorescence distributed to pixels positioned in neighboring robe cells in the image. A revised image is generated with adjusted pixel intensity values based on the deconvolution.

Other embodiments of the present invention evaluate data from the image of a hybridized microarray by modeling background noise (sometimes termed “background information” by those of skill in the art) by specifying a spatial correlation structure in the background.

In certain embodiments, the spatial correlation structure in the background is specified at the resolution of individual pixels. In other embodiments, the hybridization is summarized in terms of probe cell intensities and the spatial correlation structure of background information can be specified at the lower resolution of probe cells. In further embodiments, it is contemplated that the background can be estimated at even lower resolution where the background noise is modeled in terms of groups of probe cells forming regions by specifying a spatial correlation structure from group to group or region to region.

In particular embodiments, the statistical model includes a Markov random field to specify the distribution of configurations of background regions, where background regions can be individual pixels in the raw data, probe cells, collections of probe cells or other desired function of raw pixel data.

The analyzing step can be carried out using Gibbs sampling techniques. The results of hybridization of the probe cell locations in the image can be analyzed without considering mismatch probe sets. In addition, the analysis can be carried out independent of the sequence of the nucleic acids on the microarray.

Additional embodiments of the present invention are directed to systems for analyzing images of hybridized arrays of nucleic acid probes. The system includes a processor and computer program code for estimating background illumination in an image using a predetermined multivariate statistical model comprising at least one of a blurring kernel to deconvolute blur and a parameterized spatial model or spatial multivariate model of the background.

Other embodiments of the present invention provide microarrays having a substrate and a plurality of nucleic acid probe cells positioned on a primary surface thereof, wherein the probe cells have a hexagonal shaped perimeter.

Still other embodiments of the present invention include an array of oligonucleotide probes immobilized on a solid support. The array has a hybridization surface that is free or substantially free of mismatch probes.

Additional embodiments of the present invention include arrays of oligonucleotide probes immobilized on a solid support. The array is sized at about 1.28 cm×1.28 cm or less, and the array comprises at least about 400,000-1,000,000 individual perfect match probe cells thereon.

In certain other embodiments, the array is an array of oligonucleotide probes immobilized on a solid support. The array has a hybridization surface that is free or substantially free of mismatch probes, and the probes are sized to cover an area on the hybridization surface that is about 21.5 μm×25 μm or less per probe.

In still other embodiments of the present invention, the results of hybridization in expression probe arrays of nucleic acid probes can be evaluated by determining the intensity of a plurality of probe cells associated with perfect match probe sequences in an image of a hybridized probe array. The probe cells are ranked based on the determined intensity calculated for the perfect matches after background substraction so that ranking is carried out without regard to information from mismatch probes. The results of the hybridization are classified based on the ranking.

As will be appreciated by those of skill in the art in light of the present disclosure, embodiments of the present invention may include methods, systems and/or computer program products.

The foregoing and other objects and aspects of the present invention are explained in detail in the specification set forth below.

### BRIEF DESCRIPTION OF THE DRAWINGS

FIG. 1 is a grayscale image of log transformed intensity data of a high-density synthetic-oglionucleotide DNA microarray (HSDM).

FIGS. 2A-2D represent a 100×100 pixel region of image intensity data of the image shown in FIG. 1. FIG. 2A is an image of log transformed pixel intensities represented in grayscale according to embodiments of the present invention. FIG. 2B is an image of the same pixels in FIG. 2A shown with a surface response represented by ray tracing and pseudo coloring. FIG. 2C is a corresponding image of the spatial distribution of the estimated background pixel intensities according to embodiments of the present invention. FIG. 2D is a corresponding image illustrating pixel intensities with the estimated background intensities subtracted according to embodiments of the present invention.

FIGS. 3A-3D represent a 100×100 pixel region of an HSDM which exhibits an image artifact. FIG. 3A is a grayscale image using log transformed pixel intensities according to embodiments of the present invention. FIG. 3B shows the same pixels shown in FIG. 3A but with a surface response enhanced with the aid of ray tracing and psuedo coloring. FIG. 3C is a corresponding image of the estimated background pixel intensities which reveals a portion of the artifact according to embodiments of the present invention. FIG. 3D illustrates the pixels in the image of FIG. 3B reduced by the estimated background intensities of FIG. 3C according to embodiments of the present invention.

FIG. 4 is a flow chart of operations for analyzing an HSDM image according to embodiments of the present invention.

FIG. 5 is a flow chart of operations for analyzing the image of an expressed or hybridized microarray according to embodiments of the present invention.

FIG. 6 is a flow chart of operations for analyzing the image of a microarray according to embodiments of the present invention.

FIG. 7A is a schematic illustration of a deconvolution process for establishing the intensity of the probe cell in an image of an HSDM according to embodiments of the present invention.

FIG. 7B is a schematic illustration of a probe cell location with neighboring probe cells according to embodiments of the present invention.

FIG. 7C is a graph of estimated probe cell intensities over a one-dimensional array of 128 artificially generated probe cell intensities with the estimated background level drawn as a line across the estimated intensities according to embodiments of the present invention.

FIG. 8 is a schematic of a tiling or probe cell configuration of a microarray according to embodiments of the present invention.

FIG. 9 is an image of HDSM illustrating responding probe cells. The probe cells classified as hybridizing to RNA from an up-regulated gene in ER+ tumors are in white and probe cells classified as hybridizing to RNA from a down-regulated gene in ER+ tumors are shown in black. Unclassified probe cells are shown in gray.

FIG. 10 is an image of responding probe cell sets. Probe sets classified as hybridizing to RNA from an up-regulated gene in ER+ tumors have their perfect match and mismatch probe cells colored white. Probe sets classified as hybridizing to RNA from a down-regulated gene in ER+ tumors have their perfect match and mismatch probe cells colored black. The remaining probe cells are gray. Not all probe sets are contiguous.

FIGS. 11A and 11B are graphs of probe cell rankings. Probe cell rankings are those probe sets classified as binding RNA coincidental to ER tumor status. On the vertical axis are probe cell ranks with respect to all other perfect matches in the same observation. Moving horizontally along the graph will traverse individual perfect matches within a probe set. Red crosses indicate ranks of perfect match probe cells from ER+ tumors, black crosses are ranks of perfect match probe cells from ER-tumors. FIG. 11A is for probe set x03635 which interrogates the estrogen receptor gene for transcription, classified as up-regulated. FIG. 11B is probe set 119067 which interrogates the gene coding human nf-kappa-b transcription factor p65 for transcription, classified as down-regulated.

FIG. 12 is a schematic illustration of a system for removing background illumination influence on image intensity in an image according to embodiments of the present invention.

### DESCRIPTION OF EMBODIMENTS OF THE INVENTION

The present invention will now be described more fully hereinafter with reference to the accompanying figures, in which preferred embodiments of the invention are shown. This invention may, however, be embodied in many different forms and should not be construed as limited to the embodiments set forth herein. Like numbers refer to like elements throughout. In the figures, certain regions, components, features or layers may be exaggerated for clarity. The broken lines in the figures indicate that the feature or step so indicated is optional.

The present invention is directed at systems, methods and/or computer programs for accounting for the influence of background illumination and/or noise which may be present in the image (or digital/electronic files thereof) of a microarray to inhibit the distortion of illumination or intensity data which may be attributed to these parameters. As used herein, the term “background” includes the intensity influence of background illumination in the image associated with one or more of image acquisition, probe or chip abnormalities, manufacturing or processing defects associated with the microarray, and, hybridization or expression abnormalities or noise associated with the nucleic acid sequence on the microarray.

The microarray can be a high-density microarray, chip, or expression probe for evaluating genetic expression or hybridization in high volume parallel acquisition (such as hybridized nucleic acid probes and/or HSDM's). In a representative embodiment, the files or images reflect fluorescence data from a biological array, but the files may also represent other data such as heat activated, or radioactive intensity data. Examples of microarrays commercially available include the high-density synthetic-oglionucleotide DNA microarray from Affymetrix, Inc., discussed above, and other slides such as spotted arrays by Molecular Dynamics of Sunnyvale, Calif., Incyte Pharmaceuticals of Palo Alto, Calif., Nanogen (NanoChip) of San Diego, Calif., Protogene, of Palo Alto, Calif., Corning, of Acton, Mass. See URL gene-chips.com for information on gene expression companies.

The term “expressed” includes genetic or biomaterial which is hybridized or activated such that genetic information is optically or visually detectable and/or imageable. The genetic or biomaterial includes, but is not limited to, nucleic acids, proteins, peptides, strings of monomers, and the like, and also includes fluorescently labeled RNA which binds to DNA probes and the like. The image is typically a digital image obtained by an optical scanning system which may be visually and/or digitally presented in gray scale or color encoded intensity scales. Pixel intensity data associated with the image can be saved as an electronic and/or digital file for computational signal processing.

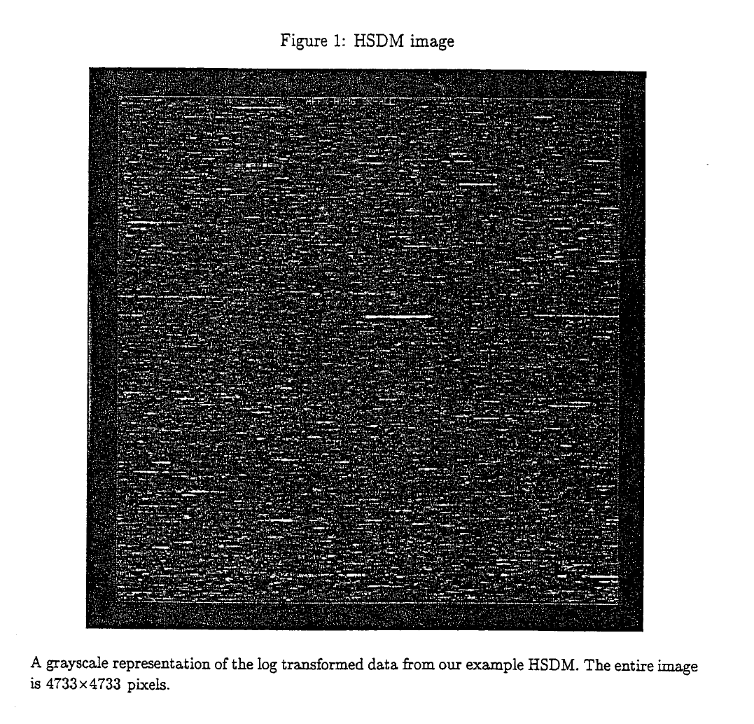

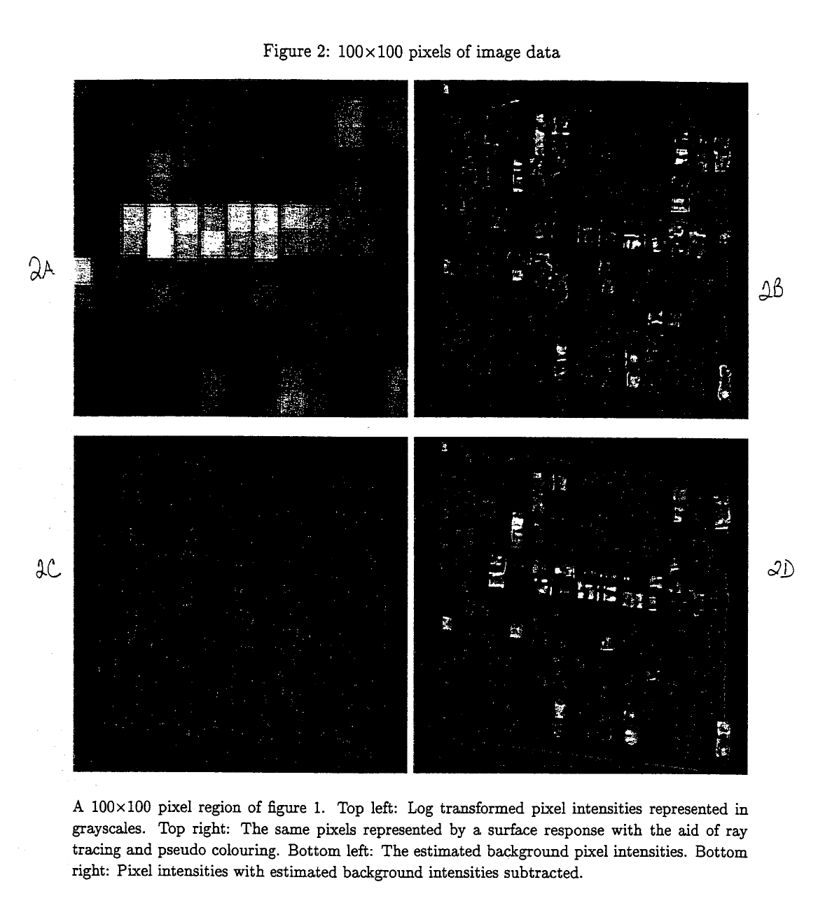

In certain embodiments, the methods, systems, and/or computer products provided by the present invention can employ a statistical model which evaluates predetermined parameters to estimate background illumination due to one or more of signal noise attributed to hybridization of target probes, background noise inherent in the process of data acquisition obtained via a scanned illuminated target, and artifacts or defects on the HSDM itself. In certain embodiments, as shown in FIGS. 2A-2D, the background estimate is generated so that it is a spatially distributed representation of the influence of the background in the image to provide a pixel-variable or pixel level resolution of the background estimate of the probe cell location in the image undergoing analysis. FIG. 2A is a grayscale image of log transformed pixel intensities of a 100×100 pixel region of FIG. 1. FIG. 2B is a representation of the surface response shown in FIG. 2A, but shown with the aid of ray tracing and pseudo coloring. FIG. 2C is an image of the estimated background in the image of FIGS. 2A and 2B. FIG. 2D is a “corrected” image where the pixel intensities shown in FIG. 2B have been subtracted by the pixel intensities shown in FIG. 2C to provide a more representative intensity distribution in the image. As shown, the background estimate can be performed such that it is spatially distributed to represent background variation within the probe cell as well as across a larger region of the image (FIG. 2C). Appropriately accounting for the influence that the background may contribute to the intensity data of the hybridized probe cells in the image may allow for more reproducible results.

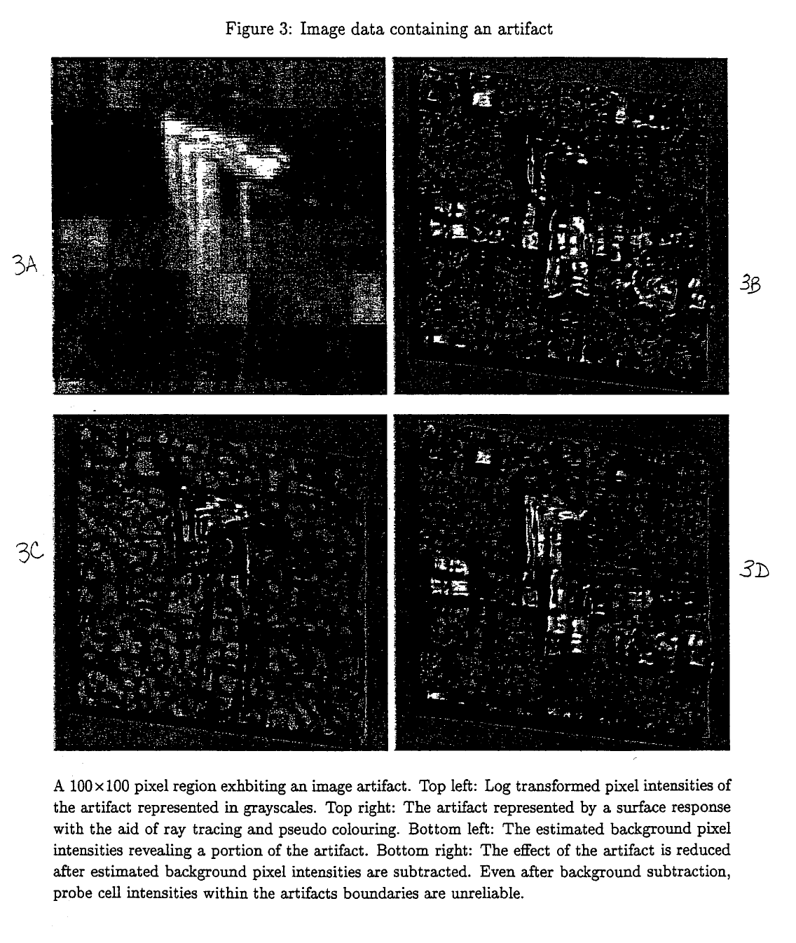

As shown in FIGS. 3A-3D, in certain embodiments, the spatially distributed background estimate can be used to automatically assess data quality and/or identify and discount those portions of the image that may contain artifacts. FIG. 3A illustrates a 100×100 pixel region exhibiting an image artifact. FIG. 3B illustrates the artifact and pixels of FIG. 3A using ray tracing and pseudo coloring. FIG. 3C shows the background estimate of the pixels of FIGS. 3A and 3B (revealing a portion of the artifact). FIG. 3D illustrates the probe cell intensities after the background estimates are subtracted. As shown in FIG. 3D, the artifact effect is reduced, but probe cell intensities within the artifact boundaries may be unreliable.

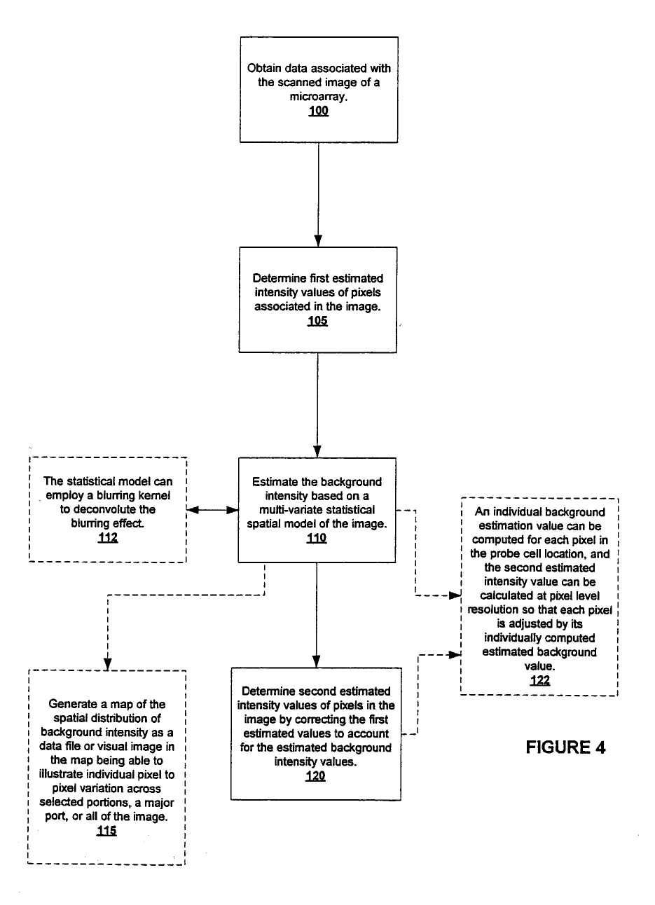

As shown in FIG. 4, operations according to embodiments of the present invention may begin by obtaining intensity data associated with a scanned image of a microarray having a plurality of probe cells thereon (block 100). A first estimated intensity value of pixels in the image can be determined (block 105). This intensity data may include both background and hybridization intensity (fluorescence or lack of fluorescence) contributions. The background intensity values of the pixels in the probe cell location can be estimated based on a predetermined multivariate statistical model of the image (block 110). The multivariate statistical model can include one or both of: (a) a blurring kernel used to deconvolute blur; and (b) a spatial multivariate model of the background. Second estimated intensity values of pixels in the image can be determined by correcting the first estimated values to account for the estimated background intensity values (block 120) so as to more closely represent the intensity of the hybridization or gene activity.

In certain embodiments, the statistical model can employ a blurring kernel to deconvolute the blurring effect (block 112) to provide better representations of features, such as probe cells. In certain embodiments, the blurring kernel can be parameterized so that the statistical model includes blurring parameters. The statistical model can include selected distributional parameters that evaluate the intensity contribution of certain features associated with background illumination to deconvolute the image to be more representative of the intensity associated with the hybridization activity of the probe cell location in the image.

In other embodiments, a map of the spatial distribution of the background intensity can be generated as a data file or visual image which can illustrate or represent individual variation pixel-to-pixel variation across selected portions, a major portion, or all of the image (block 115).

In certain embodiments, an individual background estimation value can be computed for each pixel and the second estimated intensity value can be calculated at a pixel level resolution so that each pixel can be adjusted by its individually computed estimated background value (block 122). The background intensity is, therefore, calculated based on active nucleic probes on the surface of the chip (not requiring an inactive hybridization or “blank” region).

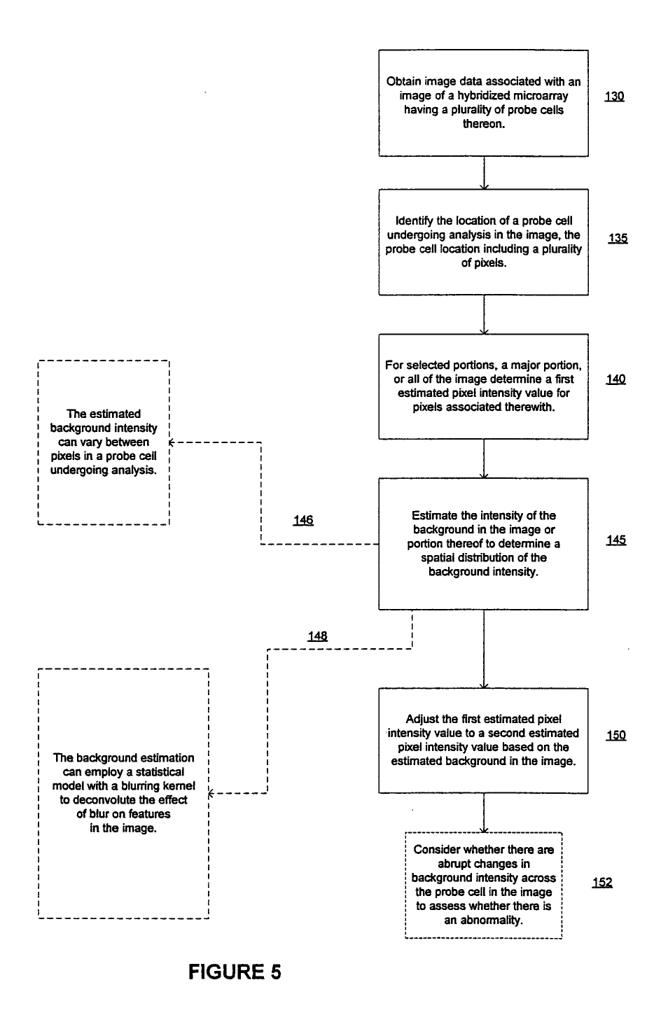

Referring now to FIG. 5, exemplary operations for analyzing an image to account for background illumination and/or noise are illustrated. As shown, an image of an expressed microarray is obtained (block 130). In certain embodiments, to establish the estimated level of background/noise in the image, the extracted raw image data at the resolution of individual pixels can be obtained and analyzed. Still referring to FIG. 5, one or more individual probe cell locations in the image may be positionally estimated in the image as desirable (block 135). The estimates of the probe cell locations may be provided in any suitable manner such as via conventional operations or as described in U.S. Pat. Nos. 6,090,555 and 5,631,734, and co-pending, co-assigned U.S. Provisional Patent Application Serial No. ______ identified by Attorney Docket No. 5405-261PR; the contents of these documents are hereby incorporated by reference as if recited in full herein.

Still referring to FIG. 5, for a selected portion, a major portion, or all, of the image, a first intensity value for each pixel associated therewith can be estimated (block 140). The intensity of the background in the image or portion thereof under analysis is estimated to determine the spatial distribution of the variation of the background intensity (block 145). In certain embodiments, the background/noise level can define the “zero” level of illumination in the scale of pixel intensity. The first estimated pixel intensities are recalculated to a second estimated value, the second estimated intensity value being adjusted (typically reduced) by the estimated background intensity (block 150). The second estimated intensity may be more representative of actual signal in the image thereby providing, in a more reproducible manner, probe cell intensities.

In certain embodiments, the background estimation can employ a statistical model which includes a blurring kernel to deconvolute the effect of blur on features in the image (block 148). The deconvolution of blur can improve estimates of the background noise, particularly in regions near the perimeter of features, such as probe cells, where the effect of blur impacts pixel intensities to greater degrees. In certain embodiments, the spatial distribution of the background intensity can be evaluated across a portion of the image to see if it is substantially constant or if there is abrupt change (pixel to pixel or region to region) to assess whether there is potential error, abnormality or artifact in the image (block 152). In certain embodiments, the background intensity can vary pixel-to-pixel across a probe cell undergoing analysis (block 146).

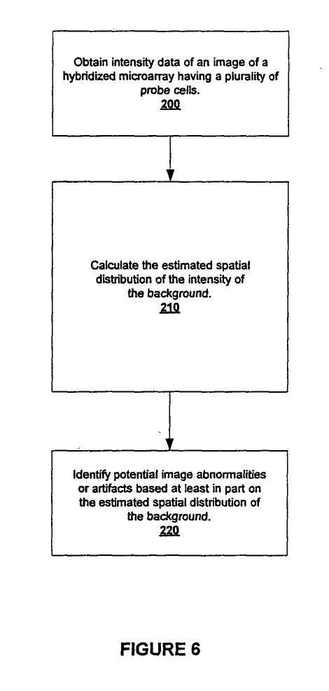

In certain other embodiments, as shown in FIG. 6, intensity data associated with the image of an expressed and/or hybridized microarray can be obtained (block 200). As before, the image data of the microarray represents a plurality of probe cells. The estimated spatial distribution of the intensity of the background in the image can be calculated (block 210). The estimated background level can be analyzed and any abnormality or artifact in the image identified or flagged (block 220) to thereby notify the researcher of a potential problem and/or inhibit the use of data for probe cells in corrupted regions of the image. Such identification can allow a researcher to identify and/or adjust for process errors in the data acquisition and/or hybridization process itself or help improve reproducibility in the results.

In certain embodiments, rather than process or analyze the recorded raw pixel intensities, the mathematical natural logarithm value of the of raw pixel intensities can be evaluated. The log transformation may stabilize the variance of pixel intensities with respect to the expected value of pixel intensity. Since the data is used to relate a gene's frequency of transcription to the strength of its detection of its transcripts, the monotonicity of the logarithm function allows the utility of relating increases or decreases in pixel intensities to changes in levels of expression. Other monotonic transformations of the data may be employed in a similar way.

Generally described, the biological microarray chip can be an array of nucleic acid probes. The chip layout or probe surface can be described as having a series of tiles which may be contiguously arranged or spaced or interspersed with alleys or gaps. Many tiling processes can be used including, but not limited to, sequence tiling, block tiling, and opt-tiling. Each tile can be associated with a single probe cell. A photolithographic process can used to mask on the desired sequence and/or tiling configuration, as is known to those of skill in the art. Additional descriptions of microarrays, lithographic methods, chip layouts, image processing and alignment methods, peptide arrays oligonucleotides and other polymer sequences, and associated processes are found in the following U.S. Pat. Nos. 5,795,716; 5,837,832; 5,856,174; 5,874,219; 6,153,743; 6,140,044; 5,856,101; 6,188,783; 6,150,147; 6,141,096; 5,959,098; 5,945,334; 6,090,555; 5,143,854; 5,384,261; 5,631,734; and 5,919,523. The contents of these patents are hereby incorporated by reference as if recited in full herein.

As noted above, the statistical model utilized to determine background intensity values can employ a blurring kernel to evaluate the blurring of the signal. The physical HSDM is a segmented object. The substrate or glass support on which the probe cells are laid out can be partitioned into a contiguous array of probe cells surrounded by a border. Thus, there is one segment for each probe cell, one segment for the border area and any point on the glass support can be considered to be either interior to a probe cell or interior to the border region surrounding the array of probe cells. Conventionally, each probe cell is square. A pixel in the scanned image of an HSDM maps to an area of the HSDM that could be either interior to a segment of the HSDM or straddle as many a four segments.

In the scanned image, there is a blurring process which takes place. During the scanning process each pixel may accumulate signal from the area of the HSDM it maps to as well as from a small surrounding region of the HSDM. By the same blurring process, each pixel may lose part of its signal to pixels nearby. Due to the discrete approximation of the HSDM, and the effect of the blurring process, the probe cells in an image of an HSDM may not be reliably viewed as a segmented collection.

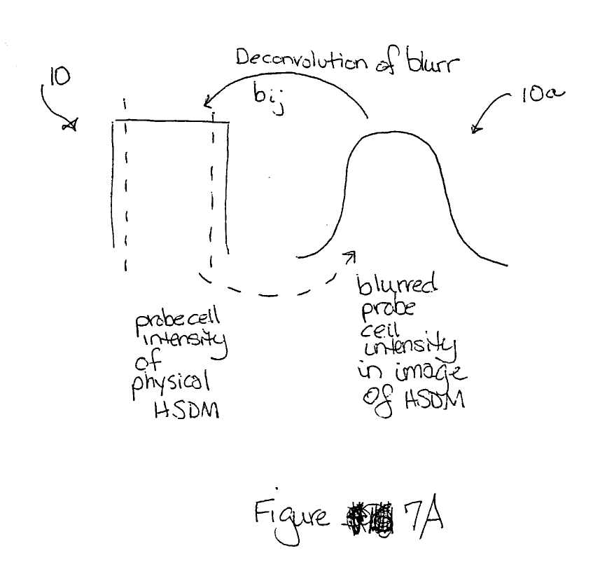

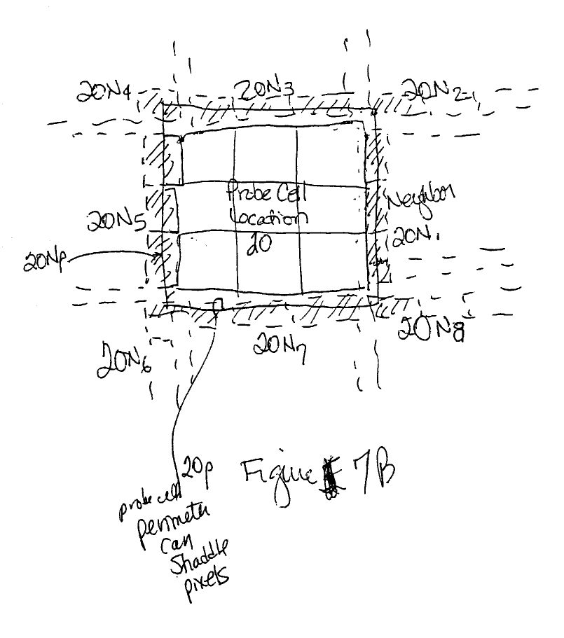

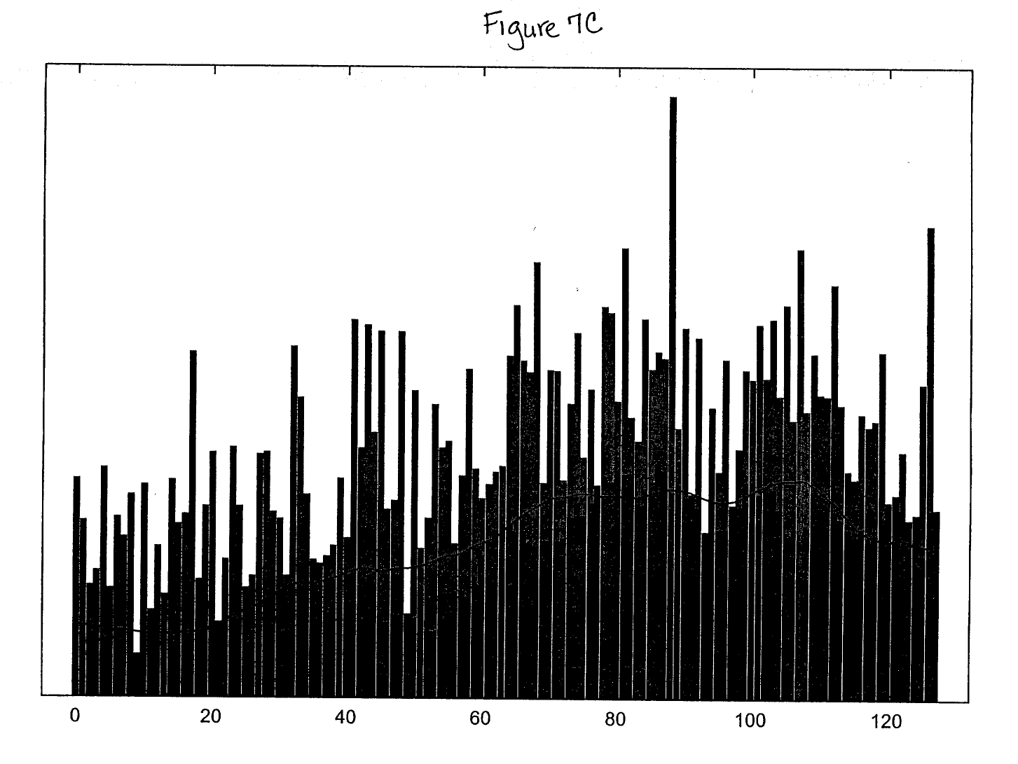

Turning now to FIG. 7A, the above blurring process and a deconvolution of the blur is illustrated. The physical or actual probe cell 10 includes a well-defined perimeter. The corresponding image of the probe cell 10a has edge portions that depart from that of the physical probe cell producing a blurred representation of the probe cell intensity in the image. The dotted lines in the actual probe cell 10 illustrate that portion of the data which may be lost or degraded in the image acquisition process.

In order to deconvolute the blur, so that the image can provide a more reliable representation of the actual probe cell in the device, certain embodiments of the present invention consider the influence of the intensity of pixels in neighboring probe cells on pixels in the probe cell location in the image undergoing analysis. FIG. 7B illustrates a probe cell location 20 with a perimeter 20p and neighboring probe cells, 20N₁, 20N₂, 20N₃, 20N₄, 20N₅, 20N₆, 20N₇, 20N₈. The perimeter of particular probe cells may straddle pixels in the image. The numbers of pixels associated with each probe cell (and its neighbors) may also be different from that shown. In certain embodiments, a blurring kernel bᵢⱼ can consider the neighborhood influence to perform the image analysis as will be discussed further below.

The estimate of background noise is independent of the nucleotide sequence presented on the surface of the microarray even though nucleotides may contribute to noise and are potential sources of error. That is, the estimation of background intensity does not require mismatch probe information. As such, the microarray does not require a “blank” cell to define the background illumination. See contra, U.S. Pat. No. 5,795,716. In certain embodiments, no pixels need be discarded from the image of the probe cell to obtain the quantification or analysis of the hybridization. See contra, U.S. Pat. No. 5,631,734. Using more of the pixels associated with the probe cells may allow the probe cell size to be reduced without losing hybridization information compared to certain conventional processes.

In certain embodiments of the present invention, the statistical model utilized to determine background intensity is a multivariate spatial model of selected distributional parameters or variables associated with the image of the probe cell. The model may include a Markov Random Field model and/or a blurring kernel to estimate background illumination. The Markov Random Field model can be implemented using Gibbs sampling techniques, the Metropolis-Hastings algorithm, or iterative conditional models. See Johnson et al., Ordinal Data Modeling, (Springer-Verlag, New York, (1999)). Distributional assumptions on parameter estimates in a model mathematical equation can be used to characterize sources of error that hinder reproducibility of observed results. Using this model, reproducibility can be studied using a single observation by taking advantage of the fact that an observation is highly-multivariate together with the spatial information in the observations elements.

As noted above, in certain embodiments a blurring kernel can be used and the hybridization analysis can be based on data from all of the pixels in a hybridization datum including those pixels on or near probe cell boundaries. The image analysis can be used to quantify and/or study gene expression on the microarray using functions of estimated probe cell intensities. Numerical estimates of uncertainty can be obtained using estimates of signal and noise parameters. As will be appreciated by those of skill in the art, the operations may be carried out on a computer using floating-point arithmetic. In operation, the implementation of the systems, methods, and operations of the present invention can be utilized to assess image quality and investigate sources that may hinder reproducibility of observations as discussed above.

In certain embodiments, probe cells may be shaped in non-conventional non-square shapes. For example, the probe cell may be shaped as a hexagon, and/or can be reduced in size. That is, unlike the conventional square shape of the probe cell, the image analysis operations of the present invention can analyze the image in a manner that: (a) may not require mismatch oligonucleotide probe information; (b) may not require that perimeter or edge portion pixels be discarded; and (c) evaluate the background and image intensity with non-square probe cells. The image analysis operations can include substantially all of the pixels in the region associated with the estimated probe cell location in the image to evaluate the results of the hybridization.

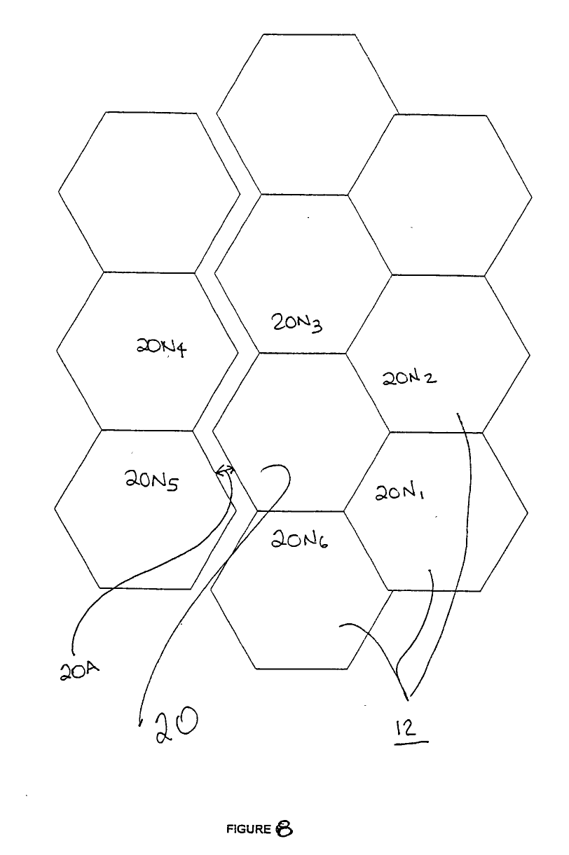

FIG. 8 illustrates one probe cell layout 12. As shown, the physical surface of the of the array can be tiled such that it includes a plurality of individual probe cells (each can define a separate probe space) selected ones, or each, having a hexagonal perimeter shape. A probe cell 20 can be analyzed so that pixels in the proximity of the border shared with neighboring probe cells are evaluated as described for FIG. 7B. The probe cell tiling 12 may be such that the individual probe cells 20 are arranged to abut the others or with alleys 20A or spaces formed therebetween, or with a mixture thereof. The hexagonal shape can reduce the perimeter size relative to the interior size of the probe cell.

As noted above, in certain embodiments, in contrast to conventional systems, the image analysis operations can reduce the number of, or eliminate the need for, mismatch probes. This may allow the conventional number of interrogation probes (such as “perfect match” probes) positioned on a single chip of similar size to be increased. On a 1.28 cm×1.28 cm chip, this number can increase from approximately 200,000 to 400,00 perfect match probes. For example, each of the probes on the chip can be sized so as to cover an area on the hybridization surface which is about 21.5 μm×25 μm or less. In certain embodiments, because fewer (or no) pixels are discarded, during the hybridization intensity analysis of the image, the size of the individual probe cells on the microarray can be reduced while maintaining a size sufficient to provide useful hybridization detection and analysis. For example, a 24 μm area square probe cell size can be reduced to an area which is below about 15 μm, and typically at about 8-12 μm. Advantageously, this can increase the number of interrogation probe cells which can be arranged on the chip (allowing increased numbers of parallel analysis).

In certain embodiments, classifying the results of hybridization in expression probe arrays of nucleic acid probes can be performed by: (a) determining the intensity of a plurality of probe cells associated with perfect match probe sequences in an image of a hybridized probe array; (b) ranking the probe cells based on the determined intensity, wherein the step of ranking is carried out without regard to information from mismatch probes; and (c) classifying the results of the hybridization based the ranking.

Generally stated, in certain embodiments, each pixel intensity may be attributed to the sum of independent contributions from: (1) fluorescently labeled RNA hybridized to probes which constitutes the signal; (2) background illumination from undetermined or environmental or set-up sources which may also be expressed as non-negative spatially correlated noise; and (3) spatially uncorrelated noise.

An example of a suitable multivariate statistical spatial model of an image of an HSDM which may be employed in image analysis according to certain embodiments of the invention will be described further below. The variable i is used to index the set of pixels in the image. For discussion purposes, this number is 4733² pixels. The variable j is used to index the set of probe cells. For discussion, this number is 536² probe cells. These numbers correspond to a microarray having 536×536 probe cells and an associated number of pixels (4733×4733). Other numbers can be used without departing from the methods, systems, and computer products of the present invention. The vector of log transformed pixel intensities is represented by “z” and individual log transformed pixel intensities are represented by “z,”. The signal of probe cell j is the total contribution of signal in z from the expressed detected probe cell signal. In this discussion, this is the fluorescently labeled RNA hybridized to probes in probe cell j. The intensity of probe cell j is the signal of probe cell j averaged over the area bounded by probe cell j on the microarray or HSDM image. The vector of probe cell intensities is written as μ=(μ₁, μ₂, . . . , μ₅₃₆²) transposed. The term “bᵢⱼ” represents the proportion of signal from probe cell j contributed to z₁. Then for each j, Σᵢbᵢⱼ=1. The vector of spatially correlated background noise is written as x, and the contribution of background to z₁ as x₁. The background vector x can be modeled as a Markov random field where the neighborhood, Nᵢ, contains the indices of the eight neighbors surrounding pixel i. The probability of observing a configuration of the background is

$$p ( x ) α {\prod }_{i} [ {\prod }_{\frac{k ∈ {N}_{i}}{k > i}} exp \{ - \frac{β}{2} {{w}_{ik} ( {x}_{i} - {x}_{k} )}^{2} \} ] , \qquad ( 1 )$$

where wᵢₖ=1 if pixel k is adjacent to pixel i in a horizontal or vertical direction, and wᵢₖ=1/\sqrt{2} if pixel k is adjacent to pixel i in a diagonal direction. The parameter β in equation (1) is not known and will be estimated. The parameter “β” which is modeled in equation (1) as a fixed quantity is not known and will be estimated. The elements of the vector of spatially uncorrelated noise, e, are assumed to be distributed identically and independently normal with mean 0 and variance τ². From the above discussion, the model equation becomes:

$${z}_{i} = {\sum }_{j} {b}_{ij} {μ}_{j} + {x}_{i} + {e}_{i} , \qquad ( 2 )$$

or equivalently,

z=Bμ+x+e. (3)

The elements of B are determined by estimates of the boundaries of the probe cells, assumptions regarding the distribution of the signal of each probe cell within its boundaries, the choice of blurring kernel and any parameters that shape or scale the kernel. “B” is the matrix containing the elements bᵢⱼ. The ith row and jth column of B contains bᵢⱼ. The estimate of the parameter β determines the smoothness of the background. Larger values of β correspond to smoother background. The estimate of the parameter “τ” determines how much uncorrelated noise is perceived to be present in the image and, thus, how precisely the observed pixel values represent signal in the presence of additive background noise. Smaller estimates of τ indicate less uncorrelated noise.

In the present example, the region covered by each probe cell can be assumed to be square. The signal of each probe cell can be assumed to be uniformly distributed within its boundaries and the blurring kernel can be assumed to be Gaussian. Due to the large number of parameters in the model and the computational difficulty involved in expediting the analysis, it may be desirable not to estimate all of the parameters jointly. Instead, in certain embodiments, a stepwise estimation procedure can be employed. First, the locations of the probe cells can be estimated and an estimated parameter τ² can be identified. Second, the width of the probe cells can be estimated as well as the blurring kernel parameters. Then the following parameters can be jointly estimated: the background configuration x, its single parameter β, and the vector, μ, of probe cell intensities using Gibbs sampling. See Johnson et al., Ordinal Data Modeling, (Springer-Verlag, New, York, (1999)).

Given that a probe cell on an HSDM maps to an area less than about 8×8 pixels in an HSDM image, an accurate estimate of B in equation (3) relies on good estimates of probe cell locations. Accurate estimates of probe cell locations are desirable for analysis of HSDM data, with or without a model for pixel intensities. As noted above, the estimated probe cell locations can be provided using the alignment techniques described in the co-pending Zuzan et al. provisional patent application incorporated by reference above. The variance of pixel intensities in a subset of pixels associated with the probe cell can be used to obtain an estimate of the τ² variable value. For example, a 5×5 grid of pixels nearest the estimated center of each probe cell can be calculated and the mean of the calculated or observed variances can be used as the estimate for τ². For an HSDM image evaluated by the model described with the 536×536 probe cell number described above, the estimate of τ² was 0.0285.

This procedure for estimating τ² does not take into account that the difference in intensities between neighboring pixels will reflect a contribution from background noise. Additional correlated noise from the background information tends to inflate τ² but this inflation is counteracted by the smoothing effect that the blurring kernel has on the uncorrelated noise. The relationship between τ² and β was investigated in the presence of a blurring kernel using simulations. From simulated data, it appears that if the true value of the product βτ² is greater than about 0.1, the estimate of τ² would not exceed twice its true value. In addition, it was observed that by analyzing simulated data, either of the estimates of β or τ may be fixed to be off by an order of magnitude and this would have little or no effect on the estimate of μ, other than the possibility of requiring longer durations of Gibbs sampling. In light of these findings, it is believed that estimates of the nuisance parameter τ² are adequate and appropriate with respect to inferences to be made about μ and that τ² need not be, but can be, estimated jointly with β.

In the present example, the probe cells can be modeled as square regions centered at their estimated coordinates with signal uniformly distributed within their boundaries. The model can be modified to account for other configurations. The possibility of gaps were allowed in an analysis between probe cells but not the possibility of probe cells overlapping. The smoothing kernel was modeled as bivariate Gaussian. The smoothing kernel was parameterized with covariance matrix σ²I. Let Fᵢ be the region of the image bounded by pixel i and let (v₁, v₂) be image coordinates within region Fᵢ. Let Gⱼ, be the region of the image which maps to probe cell j on the HSDM and let (u₁, u₂) be image coordinates within region Gⱼ. Using a Gaussian smoothing kernel, signal is distributed from (v₁, v₂) to (u₁, u₂) with probability

$$p ( {v}_{1} , {v}_{2} \vert {u}_{1} , {u}_{2} , {σ}^{2} ) = \frac{1}{2 {πσ}^{2}} exp \{ - \frac{1}{2 {σ}^{2}} [ {( {v}_{1} - {u}_{1} )}^{2} + {( {v}_{2} - {u}_{2} )}^{2} ] \} , \qquad ( 4 )$$

hence, the proportion of signal in the probe cell region Gⱼ projected onto pixel region Fᵢ, is

$${b}_{ij} = \int {\int }_{{G}_{j}} [ \int {\int }_{{F}_{i}} p ( {v}_{1} , {v}_{2} \vert {u}_{1} , {u}_{2} , {σ}^{2} ) ⅆ {v}_{1} ⅆ {v}_{2} ] ⅆ {u}_{1} ⅆ {u}_{2} . \qquad ( 5 )$$

Using equation (3), artificial images of HSDMs were generated using various combinations of kernel parameter, σ², and probe cell width. A combination of these parameters were used which generated images closely resembling the log-transformed images of the HSDMs used as initial estimates. A combination of these parameters that generated images that closely resembled the log-transformed images of the HSDM's were used as initial estimates. Real data was subsequently analyzed using equation (3) with the initial estimates of σ² and probe cell width incorporated. The results of these preliminary analyses were examined and the parameters refined. The refinements were based on choices of parameters that provided smooth transitions from probe cell to probe cell in the image of the background obtained from x. After revision, in the experimental analysis, the width of the probe cells were estimated to be 7.90 pixels and the kernel parameter, σ², was estimated to be 0.7225.

In the stepwise estimation of model parameters described above, estimations or assumptions were established regarding the locations of probe cells, probe cell boundaries, the distribution of signal within probe cells and the dispersion parameter, σ², and the smoothing kernel was assumed to be Gaussian. From these estimates all of the elements of matrix B in equation (3) can be computed. The variance σ² of the uncorrelated noise was also estimated. What remained was to estimate x along with its precision parameter β and a point estimate of μ.

In full implementation, the inclusion of prior knowledge of probe cell intensities by placing prior distributions on each μⱼ can be used. In this particular implementation, assume each μⱼ, is distributed normal with mean αⱼ and variance γ.

From equation (1), the full conditional distribution of x₁ is:

$$p ( {x}_{i} \vert β , {\{ {x}_{k} \}}_{k ∈ {N}_{i}} ) = \frac{\sqrt{β {\sum }_{k ∈ {N}_{i}} {w}_{ik}}}{\sqrt{2 π}} exp \{ - \frac{β {\sum }_{k ∈ {N}_{i}} {w}_{ik}}{2} {( {x}_{i} - \frac{{\sum }_{k ∈ {N}_{i}} {w}_{ik} {x}_{k}}{{\sum }_{k ∈ {N}_{i}} {w}_{ik}} )}^{2} \} , \qquad ( 6 )$$

which is normal with mean Σwᵢₖxₖ/Σwᵢₖ and variance 1/βΣwᵢₖ, where summation is over all kεN₁. To estimate the parameter β, a pseudo-likelihood approach based on the full conditional distribution of xᵢ in equation (6) can be used. Using equation (6) and 9 sampled values in a 3×3 region of the background, a maximum likelihood estimate of β can be obtained. At each iteration, maximum likelihood estimates for β were calculated from a sample of 1024 randomly selected 3×3 pixel regions. Each of the 1024 regions was selected from the set of all possible regions with equal probability and in each iteration a new sample was selected. The mean of these 1024 estimates was used as the estimate of β.

To estimate μ, consider that given the parameters in model equation (3), the probability can be expressed by equation (7) as:

$$p ( e \vert B , μ , x , {τ}^{2} ) α {\prod }_{i} exp \{ - \frac{1}{2 {τ}^{2}} ( {z}_{i} - {\sum }_{j} {b}_{ij} {μ}_{j} - {x}_{i} ) \} . \qquad ( 7 )$$

The right-hand side of equation (7) can be rearranged to obtain the likelihood of μⱼ. First, the right-hand side of equation (7) can be expanded to obtain

$${\prod }_{i} exp \{ - \frac{1}{2 {τ}^{2}} {( {b}_{ij} {μ}_{j} - {z}_{i} - {\sum }_{k \neq j} {b}_{ij} {μ}_{k} + {x}_{i} )}^{2} \} . \qquad ( 8 )$$

Rearranging equation (8) to separate μⱼ and multiplying the result by the prior distribution of μⱼ yields:

$$exp \{ - \frac{1}{2 {τ}^{2}} {\sum }_{i} {{b}_{ij}^{2} ( {u}_{j} - \frac{{\sum }_{i} {b}_{ij} ( {z}_{i} - {\sum }_{k \neq j} {b}_{ij} {μ}_{k} - {x}_{i} )}{{\sum }_{i} {b}_{ij}^{2}} )}^{2} \} exp \{ - \frac{1}{2 {γ}^{2}} {( {μ}_{j} - {α}_{j} )}^{2} \} , \qquad ( 9 )$$

with proportional constant terms omitted. The posterior distribution of μⱼ, in equation (9) is normal

$$N ( \frac{\frac{1}{{τ}^{2}} {\sum }_{i} {b}_{ij} ( {z}_{i} - {\sum }_{k \neq j} {b}_{ij} {μ}_{k} - {x}_{i} ) + \frac{{α}_{j}}{{γ}^{2}}}{\frac{1}{{τ}^{2}} {\sum }_{i} {b}_{ij}^{2} + \frac{1}{{γ}^{2}}} , \frac{1}{{τ}^{2}} {\sum }_{i} {b}_{ij}^{2} + \frac{1}{{γ}^{2}} ) \qquad ( 10 )$$

Prior to sampling β, x and μ, the background was initialized to z. An iteration of Gibbs sampling proceeded by estimating β using the pseudo-likelihood approach. Next the elements of x were simulated from their conditional distributions. Finally the elements of μ were simulated from their posterior distributions. The burn in period was 1000 iterations. A point estimate of the background x and a point estimate of probe cell intensities μ were estimated by the means of their simulated values over a subsequent 2000 iterations. Prior knowledge with respect to a probe cell intensity is specific to the probe sequence and RNA sample, so for this work a uniform prior distribution was employed on each αⱼ.

The image model was not concerned with the processes contributing to background noise. Instead the magnitude of the non-negative background and its correlation structure was accommodated empirically in the posterior distribution of the Markov random field. One analyst might expect the background to be smooth with gradual changes in pixel intensity while another analyst might expect the background to be an aggregate of noise contributions from a variety of sources with diverse co-variance structures. Advantageously, the neighborhood structure and estimate of β in the Markov random field can accommodate realizations of either of these expectations. After burn in, the sampled estimates of β had a mean of 8.1 which when compared to the estimate of 0.0285 for τ² suggested that the background is not smooth. (β is an estimate of precision and τ is an estimate of variance.)

An enlarged section of log transformed pixels from an example HSDM is shown in FIG. 2A accompanied by ray-traced renderings of the same section in FIGS. 2B-2C. The estimate of the background and the effect subtracting the estimated background can be seen. These images are typical of what is seen across the entire HSDM. FIG. 3A shows a region from an HSDM image containing an artifact that was partially removed after subtraction of the estimated background. Because the image of the estimated background is free of the visual impact of probe cells, artifacts are easier to identify by eye. Looking for small aberrations by visual inspection of an image that is 4733×4733 pixels is difficult. It is much easier to identify aberrations visually using the background image. Thus, in certain embodiments of the present invention, automated detection of aberrations by analyzing spatial information in the estimated background can now be provided.

Decomposition of the right-hand side of the image model in equation (3) provides an interpretation and description of the nature of reproducibility of HSDM data. Reproducibility of the biological system directly affects the estimates of μ. If a particular gene transcribes RNA in consistent quantities under a restricted set of circumstances then the reproducible behavior of that gene, with respect to observing it in the transcriptome, should be evident in μ provided that the fidelity and binding affinity of probe DNA sequences interrogating that particular gene do not become variable factors. Other errors in data acquisition, which diminish reproducibility, are found in the vector of estimated background pixel intensities x and in the second error term. For example, the artifact in FIG. 3A may be explained by a manufacturing defect. Other artifacts found during experiments cannot be as easily explained. The matrix B holds terms which affect reproducibility during post processing and analysis of extracted data. Unknowns which B depends on, such as the true form of the blurring kernel and the size and location of probe cells, each diminish reproducibility when poorly estimated. It is believed that if B is inaccurate there will be evidence in the background image indicating so.

Probe set summaries of HSDM data such as average difference and-log average ratio, which are produced by the standard software provided by Affymetrix, may obscure sources of error that hinder reproducible behavior. But from the above discussion, the image models of the present invention may more readily attribute sources of error which diminish reproducibility to the behavior of the biological system under study, the process of data acquisition (here we consider the choice of probe sequences to be part of data acquisition) and problems related modeling the extracted data found in the HSDM image. Hence the models contemplated by the present invention may be used to study reproducibility of data without empirical methods such as computing correlations and tabulating misclassifications.

The estimate of background noise permits the quality and reproducibility of individual observations to be judged without reference to any other observations. The image data can be evaluated to propose lists of regulated genes based on reproducibility alone and without claiming that gene expression was measured. By doing so, one can distinguish between reproducibility and accuracy by not relying on any numbers considered to be accurately measuring gene transcripts.

The image model can be used to establish a framework for extending large-scale parallel acquisition of gene expression data to a larger number of genes. The most obvious potential limiting factor to parallel acquisition of data is a lower bound on the size of a probe cell. As noted above, conventional analysis techniques of HSDM image analysis computes the estimate of a probe cell's intensity using a set of pixels surrounding the estimated location of the probe cell's center. On a HuGeneF1 HSDM this region is almost always 6×6 pixels, even though probe cells occupy regions that are 8×8 pixels. By discarding pixels around the perimeter of an 8×8 region, 43.75 percent of the corresponding hybridization area remains unused. As the size of a probe cell is reduced the ratio of its perimeter to its area increases and the limit of miniaturization is rapidly reached. Compounding this problem are the consequences of not accurately estimating the location of a probe cell's center and, thus, incorrectly choosing the corresponding set of pixels that best represent the probe cell's intensity. By employing a blurring kernel in our image analysis and deconvoluting the contributions of adjacent probe cells to individual pixel intensities, all of the pixels in the hybridization area of the HSDM image to be used. Stated differently, there is no need to discard the outer portion of the probe cell from the analysis.

As shown in FIG. 8, another way of reducing the ratio of probe cell perimeter to probe cell area is to pack the hybridization region with hexagonal probe cells instead of square ones. Because pixels are scanned in rows and columns, this may be counterproductive using the current process of selecting small sets of pixels to represent probe cells. But, when employing a blurring kernel, all that changes are the estimates of the elements of B in equation (3).

On another front, the prospect of discarding information from mismatch probes in the method used to find ER regulated genes as discussed below, offers a substantial opportunity to extend parallel data acquisition via the prospect of vacating half the hybridization area, thus, making room for twice as many perfect match probes and doubling the number of genes that can be interrogated for gene expression without decreasing the size of probe cells.

As noted above, the background noise can be estimated using probe cell information only, i.e., estimating the background at the resolution of the probe cells. Thus, the present invention provides methodologies for estimating the background at multiple resolutions (such as pixel, probe cell, or other portions or partial portions of the image): one particularly suitable implementation may be generated so as to be carried out at probe cell resolution. In addition, probe cell width and the blurring parameter can be estimated prior to initiating the background estimation procedures and probe cell intensities (which can be represented by the parameters identified in the right hand side of equations (2) and (3) can be estimated jointly (or concurrently)). Similarly, the probe cell locations can also be estimated concurrently. In certain embodiments, there is a reciprocal relationship where the deconvolution of blur can be incorporated into the fitting function over the fitting regions as fitting regions may overlap (such as described in co-pending and co-assigned provisional Application identified by Attorney Docket No. 5405-261PR). In certain embodiments, an estimate of the level of background noise can be obtained without scanned pixel values by using information contained in estimates of probe cell intensities that have not been corrected for background. These estimates of intensities may be obtained without using an image model. For example, in a region deemed to be interior to a probe cell, a statistic, such as the mean or 75th quantile of pixel intensity can be used as an estimate of the probe cell intensity. In order to obtain an estimate of the level of background noise in each of these probe cell summaries, the model represented by equation (3) hereinabove can be modified to operate on probe cells instead of pixels. In addition, there is generally no need to deconvolute blur level in this embodiment. The model remains a multivariate statistical spatial model and the spatial component can still be modeled as a Markov random field. Equation (11) below provides an example that can be used to represent such a model.

yⱼ=mⱼ+xⱼ+fⱼ (1)

The term “yⱼ” is the estimate of the overall intensity of probe cell “j” and “mⱼ” is the signal due to hybridization in probe cell “j”. The level of correlated background noise in probe cell “j” is shown by the term “xⱼ” and the term “fⱼ” represents the contribution from zero mean uncorrelated noise in probe cell “j”.

An example of estimating the level of background noise using only estimates of probe cell intensities can be seen in FIG. 7C. In FIG. 7C, a one-dimensional array of 128 artificially generated probe cell intensities are plotted as black and gray bars. The black or darker portion of each bar is the true intensity of the background while the lighter portion is additional signal. The line in FIG. 7C is the estimate of background noise intensity, which was obtained using the model in equation (11) as follows.

By assuming that all the values of mⱼ lie in the range d₁<mⱼ<d₂ and letting mⱼ*=(mⱼ−d₁)/(d₂−d₁), a prior distribution on mⱼ* can be assumed. The prior distribution on mⱼ* was assumed to be a Beta distribution with the probability of observing mⱼ* proportional to (1−mⱼ*)³.

The value used for d₁ was 0 and the value used for d₂ was the maximum of y₁, . . . , y₁₂₈. The vector of background noise, x₁, . . . , x₁₂₈, was modeled as a Markov random field as defined in equation (1) with weights equal to 1. The elements in the vector of uncorrelated noise, f₁, . . . , f₁₂₈, were assumed to be independently and identically distributed normal random variables with a mean of 0 and a variance of 1. The background noise in FIG. 7C was generated by simulating a Markov random field according to equation (1) with parameter β equal to 50. Rather than estimate β from the simulated noise, its known value was used to estimate the background noise. The estimate of the background noise, shown as the line in FIG. 7C was obtained by jointly simulating values for each mⱼ and yⱼ sampling these values using the Metropolis Hastings algorithm. Joint simulation mⱼ and yⱼ was used in order to avoid negative estimates of mⱼ and the Metropolis Hastings algorithm was appropriate for this joint sampling scheme where mᵢ*, and hence mⱼ, are valid on bounded intervals. A burn in period of 1000 iterations was employed, then x₁, . . . , x₁₂₈ was sampled over a subsequent 2000 iterations. The average of these 2000 post burn in period iterations is plotted as a black line in FIG. 7C.

As will be appreciated by one of skill in the art, the present invention may be embodied as a method, data or signal processing system, or computer program product. Accordingly, the present invention may take the form of an entirely hardware embodiment, an entirely software embodiment or an embodiment combining software and hardware aspects. Furthermore, the present invention may take the form of a computer program product on a computer-usable storage medium having computer-usable program code means embodied in the medium. Any suitable computer readable medium may be utilized including hard disks, CD-ROMs, optical storage devices, or magnetic storage devices.

The computer-usable or computer-readable medium may be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted or otherwise processed in a suitable manner if necessary, and then stored in a computer memory.

Computer program code for carrying out operations of the present invention may be written in an object oriented programming language such as Java®, Smalltalk, Python, or C++. However, the computer program code for carrying out operations of the present invention may also be written in conventional procedural programming languages, such as the “C” programming language or even assembly language. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

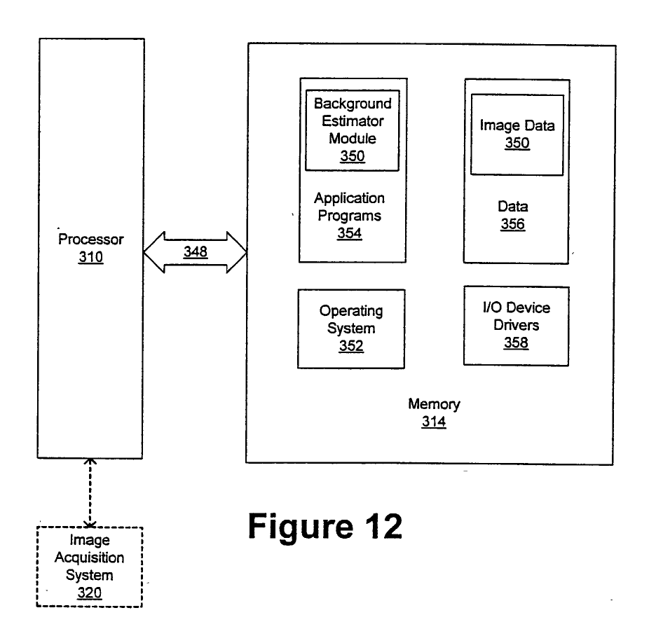

FIG. 12 is a block diagram of exemplary embodiments of data processing systems that illustrates systems, methods, and computer program products in accordance with embodiments of the present invention. The processor 310 communicates with the memory 314 via an address/data bus 348. The processor 310 can be any commercially available or custom microprocessor. The memory 314 is representative of the overall hierarchy of memory devices containing the software and data used to implement the functionality of the data processing system 305. The memory 314 can include, but is not limited to, the following types of devices: cache, ROM, PROM, EPROM, EEPROM, flash memory, SRAM, and DRAM.

As shown in FIG. 12, the memory 314 may include several categories of software and data used in the data processing system 305: the operating system 352; the application programs 354; the input/output (I/O) device drivers 358; a background estimator module 350; and the data 356. The data 356 may include image data 362 which may be obtained from an image acquisition system 320. As will be appreciated by those of skill in the art, the operating system 352 may be any operating system suitable for use with a data processing system, such as OS/2, AIX or OS/390 from International Business Machines Corporation, Armonk, N.Y., WindowsCE, WindowsNT, Windows95, Windows98 or Windows2000 from Microsoft Corporation, Redmond, Wash., PalmOS from Palm, Inc., MacOS from Apple Computer, UNIX, FreeBSD, or Linux, proprietary operating systems or dedicated operating systems, for example, for embedded data processing systems.

The I/O device drivers 358 typically include software routines accessed through the operating system 352 by the application programs 354 to communicate with devices such as I/O data port(s), data storage 356 and certain memory 314 components and/or the image acquisition system 320. The application programs 354 are illustrative of the programs that implement the various features of the data processing system 305 and preferably include at least one application which supports operations according to embodiments of the present invention. Finally, the data 356 represents the static and dynamic data used by the application programs 354, the operating system 352, the I/O device drivers 358, and other software programs that may reside in the memory 314.

While the present invention is illustrated, for example, with reference to the background estimator module 350 being an application program in FIG. 12, as will be appreciated by those of skill in the art, other configurations may also be utilized while still benefiting from the teachings of the present invention. For example, the background estimator module 350 may also be incorporated into the operating system 352, the I/O device drivers 358 or other such logical division of the data processing system 305. Thus, the present invention should not be construed as limited to the configuration of FIG. 12, which is intended to encompass any configuration capable of carrying out the operations described herein.

In certain embodiments, the background estimation module 350 includes computer program code for estimating the background illumination in the image based on a multivariate statistical model comprising at least one of: (a) a blurring kernel to deconvolute blur; and (b) a parameterized spatial model or spatial multivariate model of the background. The multivariate statistical model can be a linear additive model. The blurring kernel allows the deconvolution of the blur in the image allowing the consideration of perimeter information.

The I/O data port can be used to transfer information between the data processing system 305 and the image scanner or acquisition system 320 or another computer system or a network (e.g., the Internet) or to other devices controlled by the processor. These components may be conventional components such as those used in many conventional data processing systems, which may be configured in accordance with the present invention to operate as described herein.

While the present invention is illustrated, for example, with reference to particular divisions of programs, functions and memories, the present invention should not be construed as limited to such logical divisions. Thus, the present invention should not be construed as limited to the configuration of FIG. 12 but is intended to encompass any configuration capable of carrying out the operations described herein.

The flowcharts and block diagrams of certain of the figures herein illustrate the architecture, functionality, and operation of possible implementations of probe cell estimation means according to the present invention. In this regard, each block in the flow charts or block diagrams represents a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may in fact be executed substantially concurrently or the blocks may sometimes be executed in the reverse order, depending upon the functionality involved.

The present invention is explained further in the following non-limiting Examples.

### EXAMPLES

High-Density Synthetic-Oligonucleotide DNA Microarrays

An HSDM contains a glass support partitioned into a rectangular array of uniformly sized probe cells. Attached to the surface of each probe cell are densely packed identical sequences of synthetically manufactured oligonucleotides of single stranded DNA. With respect to the analysis here, in regard to probe cells, it is noted that: (1) the synthetic oligonucleotides within probe cells are probes that can be used to detect gene expression by hybridizing with fluorescently labeled RNA; (2) the location of a probe cell in the array can be used to determine which gene is being interrogated for expression of RNA; (3) the redundancy of the probes within each probe cell permits detection of numerous copies of RNA molecules expressed by the corresponding gene; and (4) a brightly fluorescing probe cell is indicative of a gene that was highly expressed.

For most of the work described here, a particular design called the HuGeneF1 was used. This design is made for the purpose of analyzing human gene expression. The HuGeneF1 has an array of 536×536 probe cells laid out on the surface of a glass support 1.28 cm×1.28 cm. The primary example of image analysis used here is for a single HSDM selected from a batch of 30 HSDMs used in a study of tissues extracted from breast tumors. The example selected for discussion was typical of the set of 30. There was adequate RNA hybridization and artifacts in the images were not severe enough to distract from the explanation of the statistical model of the image. This example is illustrated in FIG. 1.

The raw image scan of an HuGeneF1 HSDM is an array of 4733×4733 unsigned 16 bit grayscale pixel intensities. The potential range of pixel values was 0-65535, but in the example HSDM image the minimum pixel value was 92 and the maximum pixel value was 46207. The maximum appears to be either an upper threshold or a saturation level that was not exceeded during the scanning process. All of the data had similar minimum and maximum intensities and all lost spatial detail as the upper threshold was approached. Using the top left corner of the image as the coordinate origin and letting the first coordinate index pixels from top to bottom and the second coordinate index pixels from left to right, the corners of the array of probe cells in the example were located, by visual inspection, at the coordinates, top left (233, 242), top right (229, 4507), bottom left (4499, 254) and bottom right (4496, 4519). Between these corner positions, uniformly spaced probe cells are about 8×8 pixels in the scanned image and each would occupy a physical area of close to 21.5 μm×21.5 μm on the HSDM itself.

Relating Genes to Estrogen Receptor Status

Expression profiles of tissues extracted from two classes of breast tumors can be compared: estrogen receptor positive and negative (ER+, ER−). A monotonic relationship between the rate of transcription of a gene and the strength of detection of expression was assumed to search for genes which are consistently up-regulated or down-regulated depending on tumor ER status. The analysis provides insight into the nature of reproducibility in a manner which proceeds beyond empirical evaluations such as computations of correlations.

The analysis of the set of HSDMs which investigates gene regulation according to ER status is generally stated below. The objective was to establish a list of genes considered to be up-regulated or down-regulated depending on ER status. This process was initiated by reducing the data set from 30 observations of RNA hybridizations to 10 observations from ER+ tumor samples and 10 observations from ER-tumor samples. There were two reasons for reducing the size of the dataset: (1) clinical ER classification was uncertain for some of the tumors and observations from these were not used; and (2) in order to use the most reproducible data, it was desirable to analyze data contained in images which exhibited good RNA hybridization. Some images exhibited less than adequate RNA hybridization and these were not used. By exercising the above criteria, the dataset was limited to 10 observations from ER+ tumors. Then 10 high quality observations obtained from the ER-tumor samples were selected to provide a balanced dataset. The previously described image analysis was performed on each of the 20 HSDM observations to obtain estimates of probe cell intensities, i.e., μ, for each observation and that was our starting point.

The analysis was initially focused on individual probe cells within observations by viewing them each as possible indicators of ER status. Individual probe cells are the highest meaningful resolution at which biological response to ER status can be studied using HSDMs. Since the image model provided an estimate of background noise, mismatch probe cells were discarded and the data was able to be analyzed using only perfect match probe cells found in μ. By discarding mismatch probe cells an indicator of the extent of cross-hybridization for each perfect match was lost, but at the same time concerns regarding how accurate or consistent mismatch response actually is was dismissed. In addition, perfect matches from probe sets used as controls were discarded which left a total of 139754 perfect match probe cells drawn from 7070 probe sets on each chip.

The remainder of the analysis of genes responding to ER status is based on the following reasoning: suppose that the DNA oligonucleotide probes in a given perfect match probe cell hybridize RNA transcribed from a gene regulated by the true ER status of a tumor. Also suppose that this gene is up-regulated in ER+ tumors relative to ER− tumors. Consider how the intensity of this ER responding probe cell would rank with respect to all other perfect match probe cells in the same HSDM image. If ranking was ordered such that probe cell rank increased with probe cell intensity, this perfect match probe cell would tend to rank lower in hybridizations from ER− tumors compared to hybridizations from ER+ tumors.

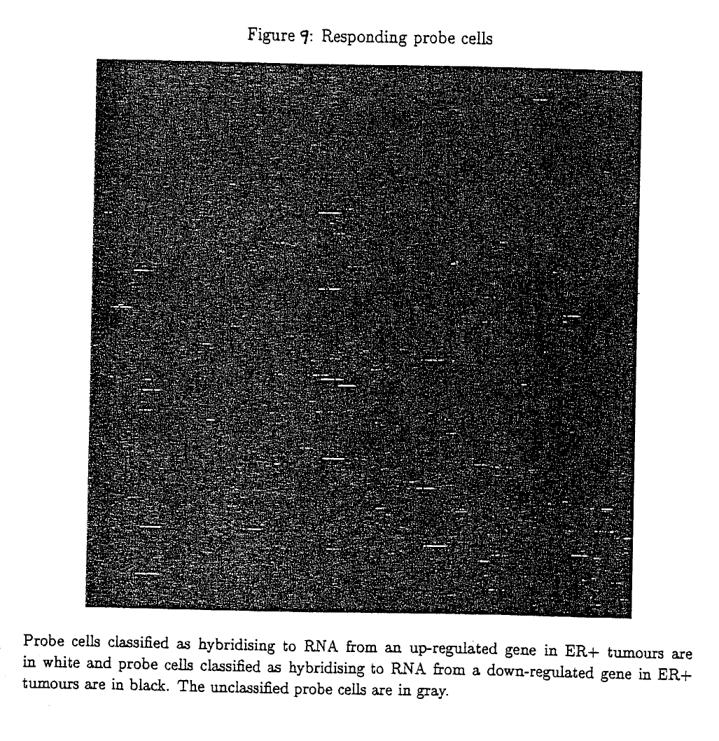

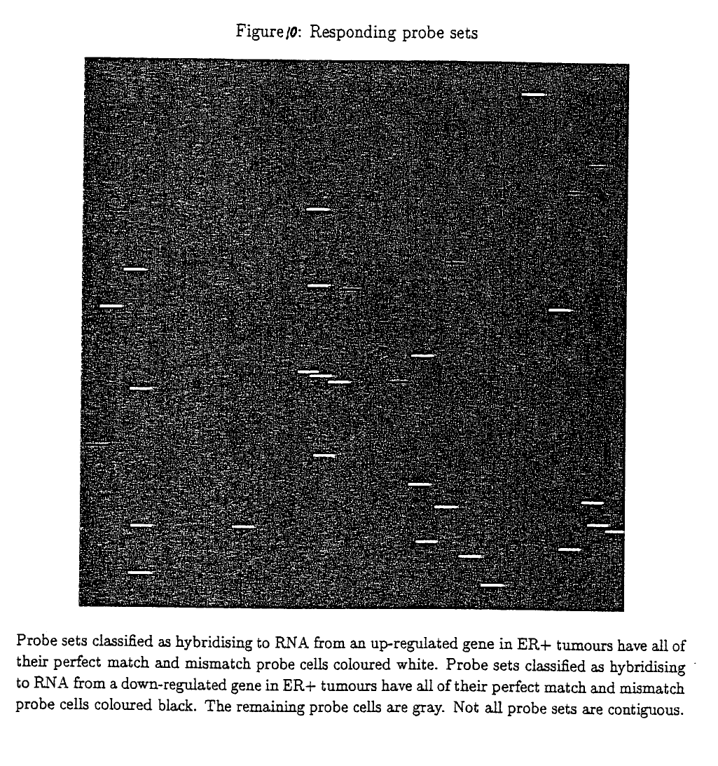

Using this reasoning, the 139754 perfect matches in each observation were ranked from lowest to highest according to estimated probe cell intensity and the perfect match probes cells were searched for ranks that consistently rose or dropped coincidental to the ER status of the observation from which they were drawn. For a given probe cell, 20 ranks will be observed, ten from each class of tumor status. If at least 9 of the 10 highest ranks were from observations obtained from ER+ tumor samples, then that probe cell was classified as hybridizing RNA from a gene that was up-regulated in ER+ tumors. Alternatively, if at least 9 of the 10 highest ranks were from observations obtained from ER− tumor samples, then that probe cell was classified as hybridizing to a gene up-regulated in ER− tumors. Under this classification scheme, a gene up-regulated with respect to one ER status will be classified as down-regulated with respect to the other ER status. FIG. 9 shows the probe cells classified as up-regulated with respect to ER+ in white and down-regulated with respect to ER+in black. To move from classifying probe cells to classifying probe sets and subsequently genes according to ER status, probe sets containing probe cells which were repeatedly classified the same were identified. For this analysis, if at least 6 perfect match probe cells in a probe set were classified the same then the probe set took on that classification and the gene that that probe set interrogates for expression was classified as up or down-regulated accordingly. In the cases where probe sets contained perfect match probe cells with opposing classifications, they cancelled each other out pair-wise and remaining perfect matches that were not cancelled out would have to support a classification if one could be made. The classified probe sets are shown in FIG. 10. Probe sets and corresponding genes classified as up-regulated in ER+ and are listed in Table 1. Down-regulated counterparts are listed in Table 2.

TABLE 1
Genes classified as up-regulated in ER+ tumors

| Identifier | Probe set/gene descriptor |
|---|---|
| d45370 | human apm2 mrna for gs2374 |
| 108044 | human intestinal trefoil factor mrna |
| 124774 | homo sapiens delta3, delta2-coa-isomerase mrna |
| M23263 | human androgen receptor mrna |
| M31627 | human x box binding protein-1 (xbp-1) mrna |
| M62403 | human insulin-like growth factor binding |
| | protein 4 (igfbp4) mrna |
| s80437 | fatty acid synthase {3′ region} [human, breast |
| | and hepg2 cells, mrna partial, 2237 nt] |
| u09770 | human cysteine-rich heart protein (hcrhp) mrna |
| u21931 | human fructose-1,6-biphosphatase (fbp1) gene |
| u22376 | c-myb gene extracted from human (c-myb) gene |
| u39840 | human hepatocyte nuclear factor-3 alpha (hnf-3 alpha) mrna |
| u41060 | human breast cancer, estrogen regulated liv-1 protein |
| | (liv-1) mrna |
| u79293 | human clone 23948 mrna sequence |
| u96113 | homo sapiens nedd-4-like ubiquitin-protein ligase wwpl mrna |
| x03635 | human mrna for oestrogen receptor |
| x12876 | human mrna fragment for cytokeratin 18 |
| x13238 | human mrna for cytochrome c oxidase subunit vic |
| x17059 | human nat1 gene for arylamine n-acetyltransferase |
| x52003 | h. sapiens ps2 protein gene |
| x53002 | human mrna for integrin beta-5 subunit |
| x55037 | h. sapiens gata-3 mrna |
| x58072 | human hgata3 mrna for trans-acting t-cell specific |
| | transcription factor |
| x70940 | h. sapiens mrna for elongation factor 1 alpha-2 |
| z23090 | h. sapiens mrna for 28 kda heat shock protein |

TABLE 2
Genes classified as down-regulated in ER+ tumors

| Identifier | Probe set/gene descriptor |
|---|---|
| 119067 | human nf-kappa-b transcription factor p65 subunit mrna |
| m13955 | human mesothelial keratin k7 (type ii) mrna |
| u04313 | human maspin mrna |
| u27185 | human rar-responsive (tig1) mrna |
| x13334 | human cd14 mrna for myelid cell-specific leucine-rich |
| | glycoprotein |
| y08374 | gp-39 cartilage protein gene extracted from |
| | h. sapiens gene encoding cartilage gp-39 protein, |
| | exon 1 and 2 (and joined cds) |

The above classification scheme can be used to account for a lack of fidelity of a probe sequence with respect to the gene it was intended to interrogate for expression. Lack of fidelity could come in two forms: (1) the probe sequence could hybridize RNA transcribed from genes other than the one intended; and (2) the probe sequence could fail to hybridize RNA from the intended gene. These two conditions could occur concurrently if the probe DNA sequence was poorly chosen. In the HuGeneF1 HSDM, probe sets are for the most part contiguous and if all the perfect matches in a probe set respond to a gene then a horizontal stripe will appear where these probe cells are located. If the classifications of probe cells in FIG. 9 are all correct, then cross hybridization occurs frequently which is evident in the many isolated probe cells that are classified as regulated according to ER status.

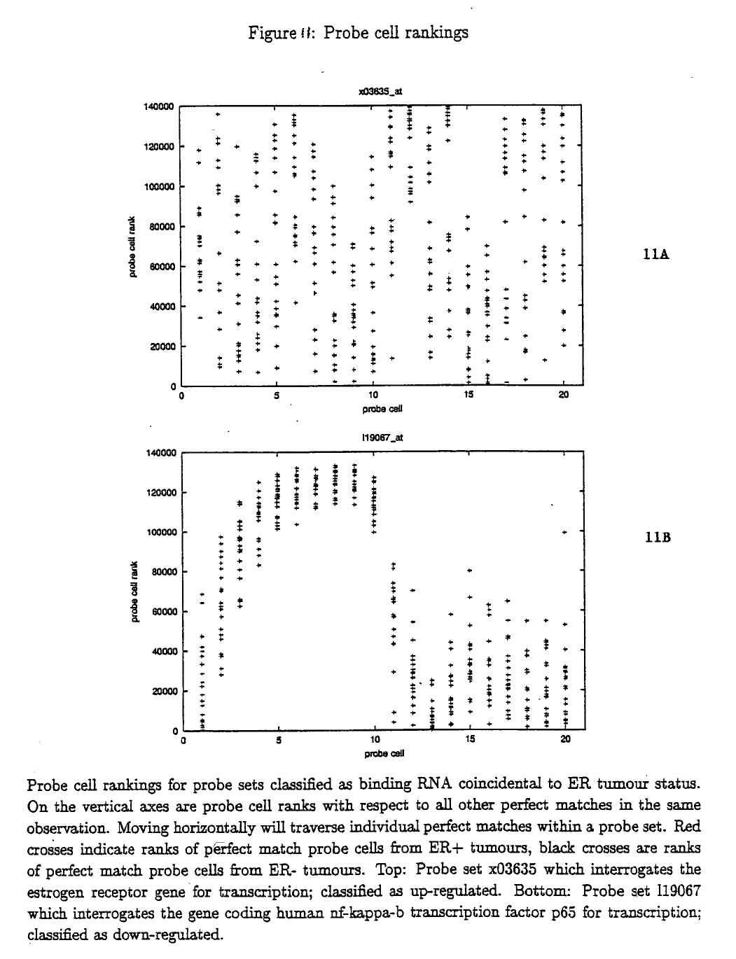

The actual rankings of perfect match probes within two probe sets are shown in FIGS. 11A and 11B. Shown in FIG. 11A, probe set x03635 which has probes designed to bind RNA transcribed from the estrogen receptor gene is obviously indicating that the estrogen receptor gene is up-regulated in ER+ tumors. Shown in FIG. 11B, probe set 119067 which has probes designed to bind RNA transcribed from the gene which codes for human nf-kappa-b transcription factor p65 indicates that this gene is down regulated in ER+ tumors but does so in a striking way. Less than half of the perfect match probe cells rank consistently coincidental to tumor status. The remainders are not discriminating. Most probe sets which were classified as corresponding to an up- or down-regulated gene had some perfect match probe cells which did not appear to be binding RNA in any consistent way, if at all. This emphasizes the importance of considering the fidelity of individual probe sequences when assessing individual probe sets and more importantly, designing probe sets. A model which analyses probe cell response from a different perspective is found in Li et al., Model-based analysis of oligonucleotide arrays: Expression index computation and outlier detection, 98 PNAS, p. 31-36 (2001).

In summary, the present invention provides image analysis methods and operations that employ at least one of: (a) a blurring kernel to deconvolute the blur in the image; and (b) a spatial multivariate model of the background. In certain embodiments, a linear additive model is used which employs both the blurring kernel and the spatial multivariate model (which may be a Markov Random field).

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A method for evaluating an image of a hybridized microarray, comprising:
obtaining an image of a microarray having a plurality of individual probe cells;
estimating the background intensity in the image based on a multivariate statistical model comprising at least one of: (a) a blurring kernel used to deconvolute blur in the image; and (b) a multivariate statistical spatial model for the background; and
determining estimated intensity values of pixels in the image by based on data from the background estimation.

2. A method according to claim 1, wherein the statistical model includes a Markov random field to model the spatial distribution of the background.

3. A method according to claim 1, wherein the statistical model includes a blurring kernel and the step of estimating considers intensity values of pixels in boundary regions of the probe cells undergoing analysis.

4. A method according to claim 2, wherein the step of estimating comprises using Gibbs sampling methods.

5. A method according to claim 2, wherein the statistical model comprises a blurring kernel used to deconvolute the image of the probe cell in the image to thereby more closely represent the intensities of the edge portions of the physical probe cell on the microarray.

6. A method according to claim 1, further comprising the step of mapping a spatial distribution of the background intensity across the image.

7. A method according to claim 1, further comprising the step of identifying an image artifact or abnormality based on the background intensity data.

8. A method according to claim 1, wherein the step of estimating is carried out on a plurality of images of different microarrays independent of a nucleotide sequence layout thereon.

9. A method according to claim 5, wherein the blurring kernel is adjusted to represent a probe cell which is not square.

10. A method according to claim 1, further comprising ranking the results of the hybridization based after the intensity values are adjusted by background data provided by the step of estimating.

11. A method according to claim 1, wherein the step of estimating comprises calculating an individual background estimation value for each pixel in at least a selected portion of the image, and said method comprises obtaining first estimated intensity values of the pixels and then calculating second adjusted estimated intensity values based on the data obtained in the step of estimating the background.

12. A method according to claim 1, wherein the step of estimating comprises logarithmically transforming individual pixel intensities.

13. A method for evaluating an image of an expressed microarray, comprising:
obtaining an image of an expressed microarray having a plurality of individual probe cells;
estimating the locations of each probe cell undergoing analysis, each probe cell location being and regions proximate thereto-defining pixels influenced by the fluorescence or lack of fluorescence of the probe cell;
determining first estimated pixel intensity values for pixels in the probe cell locations;
estimating the intensity of the background for pixels in the image to estimate a spatial distribution of the background intensity in the image; and
for each pixel in the image, reducing the first estimated pixel intensity value to a second estimated pixel intensity value based on the data provided by the estimated intensity of background.

14. A method according to claim 13, further comprising analyzing the estimated background in the image to identify an abnormality or artifact in the image.

15. A method according to claim 14, wherein the step of analyzing comprises determining whether the intensity of background illumination is substantially constant or makes an abrupt change across the probe cell in the image to assess whether there is an abnormality.

16. A method according to claim 13, wherein the step of estimating employs a multivariate spatial model of the background.

17. A method according to claim 13, wherein the step of estimating employs a blurring kernel to deconvolute the effect of blur in the image to more closely represent the features in the image.

18. A method for evaluating an image of an HSDM, comprising:
obtaining an image of an HSDM having a plurality of individual probe cells;
estimating the location of each probe cell undergoing analysis in the image, the probe cell location including a plurality of pixels;
obtaining first estimated pixel intensity values for pixels in a region associated with the probe cell in the image;
estimating background intensity for each probe cell region to obtain a spatial distribution of the background intensity in the image, wherein the step of estimating is performed such that the background intensity of the pixels can vary pixel to pixel in the image; and
determining second estimated pixel intensity values for each probe cell by reducing the first estimated pixel intensity value by its corresponding estimated background intensity.

19. A method according to claim 18, wherein the estimated background intensity value is calculated individually for each pixel in the image.

20. A method according to claim 19, wherein the step of determining the second estimated pixel intensity value comprises subtracting its corresponding estimated background value from the corresponding first estimated intensity value to generate an image adjusted at pixel level resolution for background contributions.

21. A method according to claim 18, wherein the step of estimating employs a blurring kernel to deconvolute the blur in the image.

22. A method according to claim 18, wherein the step of estimating employs a predetermined multivariate statistical spatial model which considers distributional parameters which contribute to background in the image.

23. A method according to claim 22, wherein the statistical model comprises a Markov random field.

24. A method of analyzing data obtained from an image of an hybridized microarray, comprising:
analyzing image data by using a blurring kernel to deconvolute the blurred probe cells in the image to more closely represent the intensity of the fluorescence over the entire probe cell; and
generating a revised image with adjusted pixel intensity values based on the analysis of the image data.

25. A method according to claim 24, further comprising estimating the background intensity by using a spatial multivariate model of the background.

26. A method according to claim 25, further comprising adjusting the intensity values of pixels in the image based on data obtained by the background estimation and evaluating the results of hybridization of the probe cell locations in the image without considering mismatch probe sets.

27. A method according to claim 25, wherein the step of analyzing is carried out independent of the sequence of the nucleotides on the microarray.

28. A computer program product for analyzing an image of a hybridized nucleic acid microarray chip, the computer program product comprising:
a computer readable storage medium having computer readable program code embodied in said medium, said computer-readable program code comprising:
computer readable program code that obtains data of an image of the intensities of a hybridized microarray having a plurality of individual probe cells;
computer readable program code that determines first estimated intensity values of pixels in the image;
computer readable program code that calculates estimates the intensity of background for pixels in the image based on at least one of: (a) a multivariate statistical spatial model of the background; and (b) a blurring kernel to deconvolute the blurring in the image; and
computer readable program code that determines second estimated intensity values of pixels in the image by correcting the first estimated values based on data obtained by the estimated intensity of background.

29. A computer program product according to claim 28, wherein said computer program code for rendering the multivariate statistical spatial model includes a Markov random field.

30. A computer program product according to claim 28, wherein said computer program product further comprises computer readable program code for logarithmically transforming the intensity data of the pixels.

31. A computer program product according to claim 28, wherein said computer program product for calculating estimates the intensity of background illumination comprises both the blurring kernel and the multivariate statistical spatial model.

32. A computer program product according to claim 31, wherein said computer readable program code for the blurring kernel revises the image intensity data of the image to more closely represent the intensities of the entire probe cell.

33. A computer program product according to claim 31, wherein said computer program product further comprises computer readable program code for electronically mapping a spatial distribution of the background across the image.

34. A computer program product according to claim 31, wherein said computer program product further comprises computer readable program code for identifying an image artifact or abnormality.

35. A computer program product for analyzing data representing an image of a hybridized nucleic acid microarray chip, the computer program product comprising:
a computer readable storage medium having computer readable program code embodied in said medium, said computer-readable program code comprising:
computer readable program code that obtains intensity data of an image of a hybridized microarray having a plurality of probe cells; and
computer readable program code that calculates an estimated spatial distribution of the intensity of the background in the image, the estimated intensity of the background being determined based on pixels in the image which correspond to locations on the array which have active nucleic acid probes.

36. A system for analyzing images of hybridized arrays of nucleic acid probes, comprising:
a processor; and
means for estimating background in an image using a predetermined spatial multivariate statistical model for the background.

37. A microarray having a substrate and a plurality of nucleic acid probe cells positioned on a primary surface thereof, wherein said probe cells have a hexagonal shaped perimeter.

38. An array of oligonucleotide probes immobilized on a solid support, wherein said array has a hybridization surface which is substantially free of mismatch probes.

39. An array of oligonucleotide probes immobilized on a solid support, wherein said array is sized at about 1.28 cm×1.28 cm or less, and wherein said array comprises at least about 400,000 individual perfect match probe cells thereon.

40. An array of oligonucleotide probes according to claim 39, wherein said array has a hybridization surface which is substantially free of mismatch probes, and wherein each of said probes are sized to cover an area on the hybridization surface which is about 21.5 μm×25 μm or less.

41. An array of oligonucleotide probes immobilized on a solid support, wherein said array has a hybridization surface which is substantially free of mismatch probes, and wherein said probes are sized to cover an area on the hybridization surface which is about 21.5 μm×21.5 μm or less.

42. An array according to claim 41, wherein said array comprises at least about 400,000 individual perfect match probe cells thereon.

43. A method of classifying the results of hybridization in expression probe arrays of nucleic acid probes, comprising:
estimating the background intensity in an image of a hybridized microarray using at least one of a blurring kernel to deconvolute blur in the image and a spatial multivariate model for the background;
adjusting the image intensity based on data provided by said estimating step;
ranking the probe cells based on the adjusted intensity, wherein said step of ranking is carried out without regard to information from mismatch probes; and
classifying the results of the hybridization based on said step of ranking.
